(19) United States
(12) Patent Application Publication
(10) Pub. No.: US 2014/0194874 A1
(43) Pub. Date: Jul. 10, 2014

(54) ELECTROSURGICAL END EFFECTOR WITH INDEPENDENT CLOSURE FEATURE AND BLADE

(71) Applicant: Ethicon Endo-Surgery, Inc., Cincinnati, OH (US)

(72) Inventors: Timothy G. Dietz, Terrace Park, OH (US); Mary E. Mootoo, Cincinnati, OH (US); David A. Witt, Maineville, OH (US); Zhifan F. Huang, Mason, OH (US); Jeffrey L. Aldridge, Lebanon, OH (US); Geoffrey S. Strobl, Williamsburg, OH (US); David K. Norvell, Monroe, OH (US); Jerome R. Morgan, Cincinnati, OH (US); Raymond M. Banks, Cupertino, CA (US)

(21) Appl. No.: 13/738,329

(22) Filed: Jan. 10, 2013

(51) Int. Cl. A61B 18/14

(52) U.S. Cl. CPC A61B 18/1447
USPC 606/45

(57) ABSTRACT

An apparatus includes a body, an end effector, and outer beam, and an inner beam. The body comprises an actuator. The end effector is in communication with the body and has a first jaw and a second jaw. The outer beam is able to advance within the end effector such that the outer beam closes the second jaw toward the first jaw. The advancement of the outer beam is controlled mainly by the actuator. The inner beam is also able to advance within the end effector such that the inner beam transects tissue. The actuator is operable to control the advancement of the inner beam in at least two stages. The actuator advances the inner beam and the outer beam together in a first state. The actuator advances the inner beam while the outer beam remains stationary in a second stage.

### BACKGROUND

A variety of surgical instruments include a tissue cutting element and one or more elements that transmit RF energy to tissue (e.g., to coagulate or seal the tissue). An example of such a device is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein; U.S. Pat. No. 7,112,201 entitled “Electrosurgical Instrument and Method of Use,” issued Sep. 26, 2006, the disclosure of which is incorporated by reference herein; U.S. Pat. No. 7,125,409, entitled “Electrosurgical Working End for Controlled Energy Delivery,” issued Oct. 24, 2006, the disclosure of which is incorporated by reference herein; U.S. Pat. No. 7,169,146 entitled “Electrosurgical Probe and Method of Use,” issued Jan. 30, 2007, the disclosure of which is incorporated by reference herein; U.S. Pat. No. 7,186,253, entitled “Electrosurgical Jaw Structure for Controlled Energy Delivery,” issued Mar. 6, 2007, the disclosure of which is incorporated by reference herein; U.S. Pat. No. 7,189,233, entitled “Electrosurgical Instrument,” issued Mar. 13, 2007, the disclosure of which is incorporated by reference herein; U.S. Pat. No. 7,220,951, entitled “Surgical Sealing Surfaces and Methods of Use,” issued May 22, 2007, the disclosure of which is incorporated by reference herein; U.S. Pat. No. 7,309,849, entitled “Polymer Compositions Exhibiting a PTC Property and Methods of Fabrication,” issued Dec. 18, 2007, the disclosure of which is incorporated by reference herein; U.S. Pat. No. 7,311,709, entitled “Electrosurgical Instrument and Method of Use,” issued Dec. 25, 2007, the disclosure of which is incorporated by reference herein; U.S. Pat. No. 7,354,440, entitled “Electrosurgical Instrument and Method of Use,” issued Apr. 8, 2008, the disclosure of which is incorporated by reference herein; U.S. Pat. No. 7,381,209, entitled “Electrosurgical Instrument,” issued Jun. 3, 2008, the disclosure of which is incorporated by reference herein.

Additional examples of electrosurgical cutting instruments and related concepts are disclosed in U.S. Pub. No. 2011/0087218, entitled “Surgical Instrument Comprising First and Second Drive Systems Actuatable by a Common Trigger Mechanism,” published Apr. 14, 2011, the disclosure of which is incorporated by reference herein; U.S. Pub. No. 2012/0116379, entitled “Motor Driven Electrosurgical Device with Mechanical and Electrical Feedback,” published May 10, 2012, the disclosure of which is incorporated by reference herein; U.S. Pub. No. 2012/0078243, entitled “Control Features for Articulating Surgical Device,” published Mar. 29, 2012, the disclosure of which is incorporated by reference herein; U.S. Pub. No. 2012/0078247, entitled “Articulation Joint Features for Articulating Surgical Device,” published Mar. 29, 2012, the disclosure of which is incorporated by reference herein; U.S. patent application Ser. No. 13/622,729, entitled “Surgical Instrument with Multi-Phase Trigger Bias,” filed Sep. 19, 2012, the disclosure of which is incorporated by reference herein; and U.S. patent application Ser. No. 13/622,735, entitled “Surgical Instrument with Contained Dual Helix Actuator Assembly,” filed Sep. 19, 2012, the disclosure of which is incorporated by reference herein.

While several medical devices have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side elevational view of an exemplary electrosurgical medical instrument;

FIG. 2 depicts a perspective view of the end effector of the instrument of FIG. 1, in an open configuration;

FIG. 3 depicts another perspective view of the end effector of the instrument of FIG. 1, in an open configuration;

FIG. 4 depicts a cross-sectional end view of the end effector of FIG. 2, in a closed configuration and with the blade in a distal position;

FIG. 5A depicts a side, partially cross sectional view of an alternative version of an electrosurgical instrument with a two part closing mechanism;

FIG. 5B depicts a side, partially cross sectional view of the electrosurgical instrument of FIG. 5A with the outer beam and inner beam advanced;

FIG. 5C depicts a side, partially cross sectional view of the electrosurgical instrument of FIG. 5A with the inner beam advanced while the outer beam stays stationary;

FIG. 6 depicts a perspective, cross sectional view of the electrosurgical instrument of FIG. 5A taken along the line 6-6 of FIG. 5A;

FIG. 7 depicts a flowchart view of an exemplary use of the electrosurgical instrument of FIG. 5A;

FIG. 8A depicts a side, cross sectional view of an exemplary alternative version of an end effector of an electrosurgical instrument with a pawl feature;

FIG. 8B depicts a side, cross sectional view of the end effector of FIG. 8A with the outer beam and inner beam advanced;

FIG. 8C depicts a side, cross sectional view of the end effector of FIG. 8A with the pawl feature pivoting upward and the inner beam retracted;

FIG. 8D depicts a side, cross sectional view of the end effector of FIG. 8A with the inner beam advanced and the pawl feature entering a pawl pocket;

FIG. 9 depicts a perspective view of the inner beam and outer beam of FIG. 8A;

FIG. 10A depicts a perspective, partially cross sectional view of an exemplary alternative version of an end effector with a detent feature;

FIG. 10B a perspective, partially cross sectional view of the end effector of FIG. 10A clamped and energized with the inner beam advanced and disengaging the detent feature;

FIG. 11 depicts an enlarged, perspective, cross sectional view of the detent feature of FIG. 9;

FIG. 12A depicts a side, cross sectional view of the end effector of FIG. 9 showing the ramp feature;

FIG. 12B depicts a side, cross sectional view of the end effector of FIG. 9 showing the outer beam and inner beam advanced with the inner beam descending the ramp feature and disengaging the detent feature;

FIG. 12C depicts a side, cross sectional view of the end effector of FIG. 9 showing the inner beam advanced independently of the outer beam;

FIG. 13A depicts a side, cross sectional view of an exemplary alternative version of an end effector of an electrosurgical instrument with a two-piece closure mechanism;

FIG. 13B depicts a side, cross sectional view of the end effector of FIG. 13A with the firing beam advanced and the outer driver deflecting upward to advance within the driver channel; and

FIG. 13C depicts a side, cross sectional view of the end effector of FIG. 13A with the firing beam fully advanced and the outer driver fully advanced within the driver channel.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Electrosurgical Device with Articulation Feature

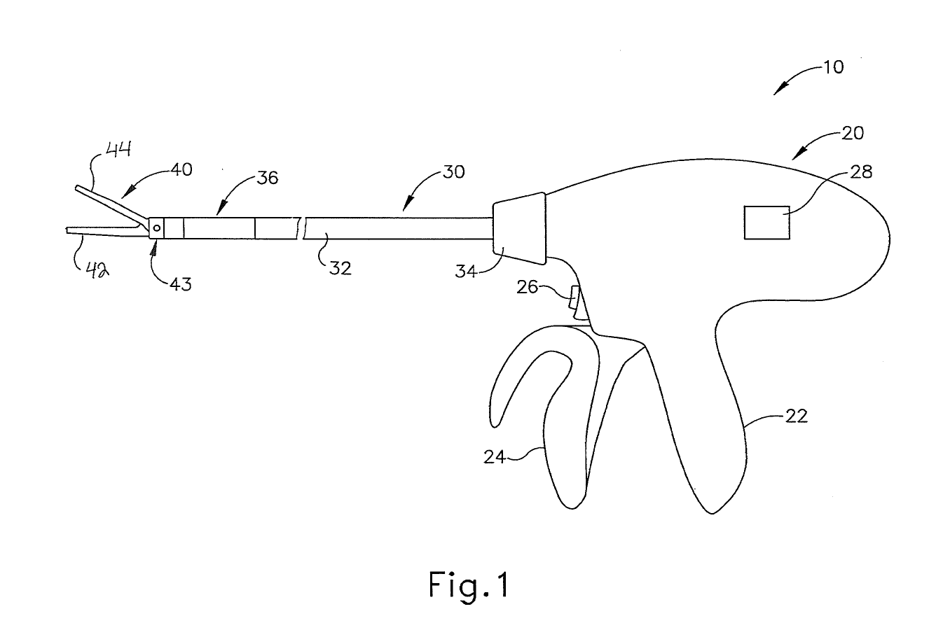

FIGS. 1-4 show an exemplary electrosurgical instrument (10) that is constructed and operable in accordance with at least some of the teachings of U.S. Pat. No. 6,500,176; U.S. Pat. No. 7,112,201; U.S. Pat. No. 7,125,409; U.S. Pat. No. 7,169,146; U.S. Pat. No. 7,186,253; U.S. Pat. No. 7,189,233; U.S. Pat. No. 7,220,951; U.S. Pat. No. 7,309,849; U.S. Pat. No. 7,311,709; U.S. Pat. No. 7,354,440; U.S. Pat. No. 7,381,209; U.S. Pub. No. 2011/0087218; U.S. Pub. No. 2012/0116379; U.S. Pub. No. 2012/0078243; U.S. Pub. No. 2012/0078247; U.S. patent application Ser. No. 13/622,729; and/or U.S. patent application Ser. No. 13/622,735. As described therein and as will be described in greater detail below, electrosurgical instrument (10) is operable to cut tissue and seal or weld tissue (e.g., a blood vessel, etc.) substantially simultaneously. In other words, electrosurgical instrument (10) operates similar to an endocutter type of stapler, except that electrosurgical instrument (10) provides tissue welding through application of bipolar RF energy instead of providing lines of staples to join tissue. It should also be understood that electrosurgical instrument (10) may have various structural and functional similarities with the ENSEAL® Tissue Sealing Device by Ethicon Endo-Surgery, Inc., of Cincinnati, Ohio. Furthermore, electrosurgical instrument (10) may have various structural and functional similarities with the devices taught in any of the other references that are cited and incorporated by reference herein. To the extent that there is some degree of overlap between the teachings of the references cited herein, the ENSEAL® Tissue Sealing Device by Ethicon Endo-Surgery, Inc., of Cincinnati, Ohio, and the following teachings relating to electrosurgical instrument (10), there is no intent for any of the description herein to be presumed as admitted prior art. Several teachings below will in fact go beyond the scope of the teachings of the references cited herein and the ENSEAL® Tissue Sealing Device by Ethicon Endo-Surgery, Inc., of Cincinnati, Ohio.

A. Exemplary Handpiece and Shaft

Electrosurgical instrument (10) of the present example includes a handpiece (20), a shaft (30) extending distally from handpiece (20), and an end effector (40) disposed at a distal end of shaft (30). Handpiece (20) of the present example includes a pistol grip (22), a pivoting trigger (24), an activation button (26), and an articulation control (28). Trigger (24) is pivotable toward and away from pistol grip (22) to selectively actuate end effector (40) as will be described in greater detail below. Activation button (26) is operable to selectively activate RF circuitry that is in communication with end effector (40), as will also be described in greater detail below. In some versions, activation button (26) also serves as a mechanical lockout against trigger (24), such that trigger (24) cannot be fully actuated unless button (26) is being pressed simultaneously. Examples of how such a lockout may be provided are disclosed in one or more of the references cited herein. In addition or in the alternative, trigger (24) may serve as an electrical and/or mechanical lockout against button (26), such that button (26) cannot be effectively activated unless trigger (24) is being squeezed simultaneously. It should be understood that pistol grip (22), trigger (24), and button (26) may be modified, substituted, supplemented, etc. in any suitable way, and that the descriptions of such components herein are merely illustrative.

Shaft (30) of the present example includes an outer sheath (32) and an articulation section (36). Articulation section (36) is operable to selectively position end effector (40) at various angles relative to the longitudinal axis defined by sheath (32). Articulation section (36) of shaft (30) may take a variety of forms. By way of example only, articulation section (36) may be configured in accordance with one or more teachings of U.S. Pub. No. 2012/0078247, the disclosure of which is incorporated by reference herein. As another merely illustrative example, articulation section (36) may be configured in accordance with one or more teachings of U.S. Pub. No. 2012/0078248, entitled “Articulation Joint Features for Articulating Surgical Device,” published Mar. 29, 2012, the disclosure of which is incorporated by reference herein. Various other suitable forms that articulation section (36) may take will be apparent to those of ordinary skill in the art in view of the teachings herein. It should also be understood that some versions of instrument (10) may simply lack articulation section (36).

In some versions, shaft (30) is also rotatable about the longitudinal axis defined by sheath (32), relative to handpiece (20), via a knob (34). Such rotation may provide rotation of end effector (40) and shaft (30) unitarily. In some other versions, knob (34) is operable to rotate end effector (40) without rotating any portion of shaft (30) that is proximal of articulation section (36). As another merely illustrative example, electrosurgical instrument (10) may include one rotation control that provides rotatability of shaft (30) and end effector (40) as a single unit; and another rotation control that provides rotatability of end effector (40) without rotating any portion of shaft (30) that is proximal of articulation section (36). Other suitable rotation schemes will be apparent to those of ordinary skill in the art in view of the teachings herein. Of course, rotatable features may simply be omitted if desired.

Articulation control (28) of the present example is operable to selectively control articulation section (36) of shaft (30), to thereby selectively position end effector (40) at various angles relative to the longitudinal axis defined by shaft (30). By way of example only, some merely illustrative forms that articulation control (28) and other components of handpiece (20) may take are disclosed in U.S. Pub. No. 2012/0078243, the disclosure of which is incorporated by reference herein; in U.S. Pub. No. 2012/0078244, entitled “Control Features for Articulating Surgical Device,” published Mar. 29, 2012, the disclosure of which is incorporated by reference herein; and in U.S. patent application Ser. No. 13/622,735, the disclosure of which is incorporated by reference herein. Still other suitable forms that articulation control (28) may take will be apparent to those of ordinary skill in the art in view of the teachings herein. It should also be understood that some versions of instrument (10) may simply lack an articulation control (28).

B. Exemplary End Effector

End effector (40) of the present example comprises a first jaw (42) and a second jaw (44). In the present example, first jaw (42) is substantially fixed relative to shaft (30); while second jaw (44) pivots relative to shaft (30), toward and away from first jaw (42). In some versions, actuators such as rods or cables, etc., may extend through sheath (32) and be joined with second jaw (44) at a pivotal coupling (43), such that longitudinal movement of the actuator rods/cables/etc. through shaft (30) provides pivoting of second jaw (44) relative to shaft (30) and relative to first jaw (42). Of course, jaws (42, 44) may instead have any other suitable kind of movement and may be actuated in any other suitable fashion. By way of example only, and as will be described in greater detail below, jaws (42, 44) may be actuated and thus closed by longitudinal translation of a firing beam (60), such that actuator rods/cables/etc. may simply be eliminated in some versions.

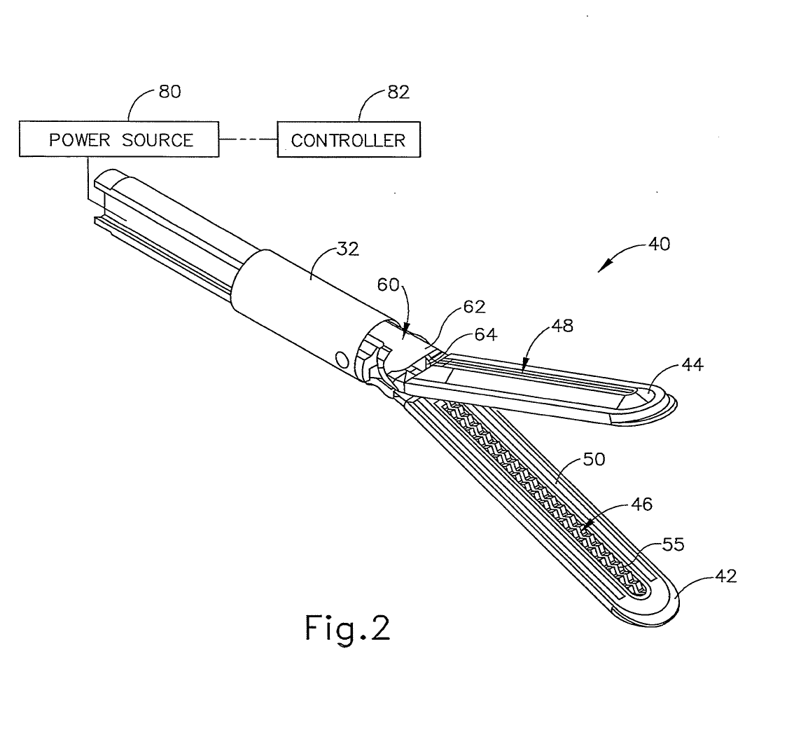

As best seen in FIGS. 2-4, first jaw (42) defines a longitudinally extending elongate slot (46); while second jaw (44) also defines a longitudinally extending elongate slot (48). In addition, the top side of first jaw (42) presents a first electrode surface (50); while the underside of second jaw (44) presents a second electrode surface (52). Electrode surfaces (50, 52) are in communication with an electrical source (80) via one or more conductors (not shown) that extend along the length of shaft (30). Electrical source (80) is operable to deliver RF energy to first electrode surface (50) at a first polarity and to second electrode surface (52) at a second (opposite) polarity, such that RF current flows between electrode surfaces (50, 52) and thereby through tissue captured between jaws (42, 44). In some versions, firing beam (60) serves as an electrical conductor that cooperates with electrode surfaces (50, 52) (e.g., as a ground return) for delivery of bipolar RF energy captured between jaws (42, 44). Electrical source (80) may be external to electrosurgical instrument (10) or may be integral with electrosurgical instrument (10) (e.g., in handpiece (20), etc.), as described in one or more references cited herein or otherwise. A controller (82) regulates delivery of power from electrical source (80) to electrode surfaces (50, 52). Controller (82) may also be external to electrosurgical instrument (10) or may be integral with electrosurgical instrument (10) (e.g., in handpiece (20), etc.), as described in one or more references cited herein or otherwise. It should also be understood that electrode surfaces (50, 52) may be provided in a variety of alternative locations, configurations, and relationships.

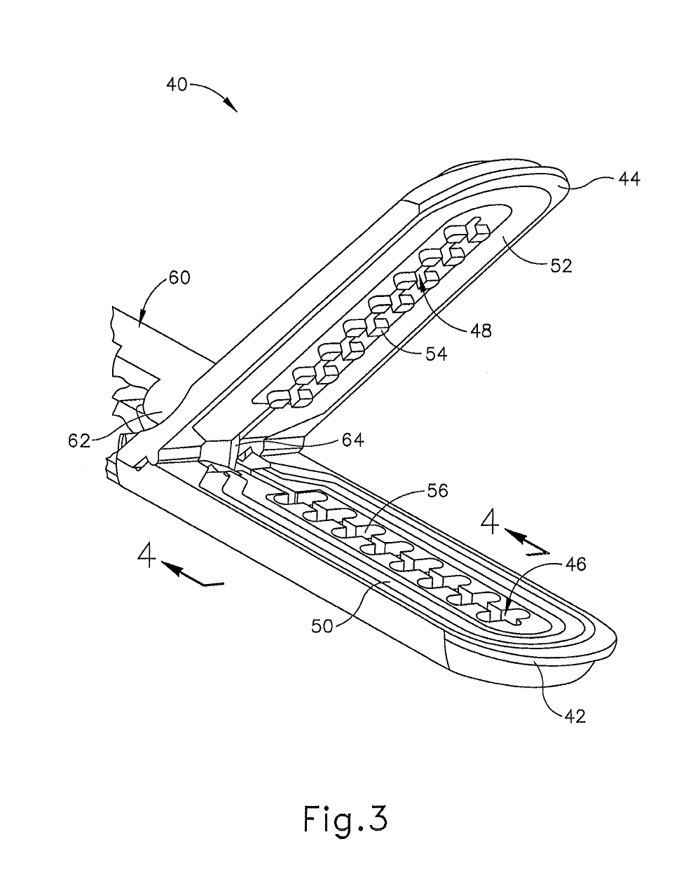

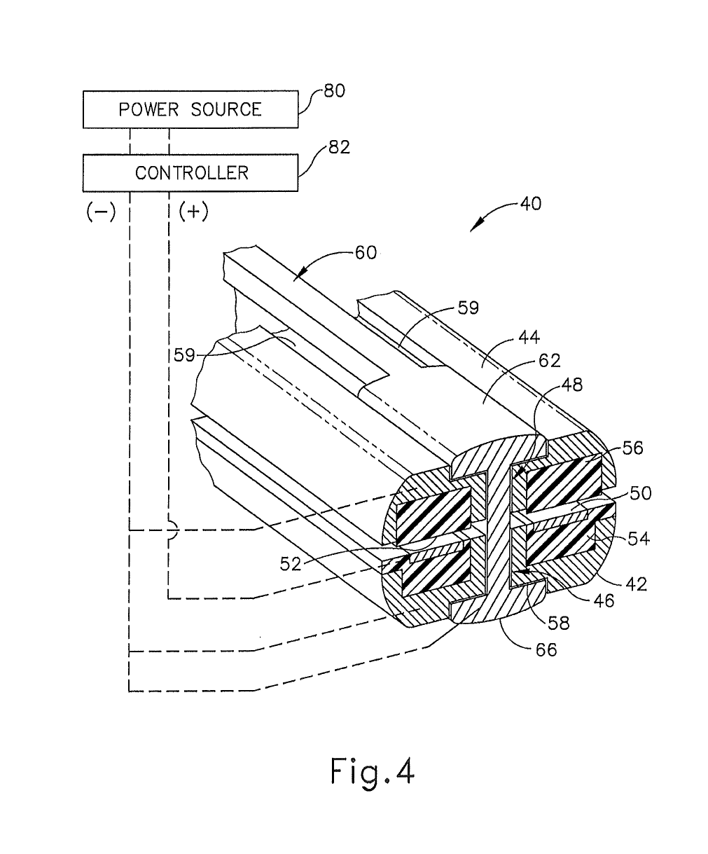

As best seen in FIG. 4, the lower side of first jaw (42) includes a longitudinally extending recess (58) adjacent to slot (46); while the upper side of second jaw (44) includes a longitudinally extending recess (59) adjacent to slot (48). FIG. 2 shows the upper side of first jaw (42) including a plurality of teeth serrations (46). It should be understood that the lower side of second jaw (44) may include complementary serrations that nest with serrations (46), to enhance gripping of tissue captured between jaws (42, 44) without necessarily tearing the tissue. FIG. 3 shows an example of serrations (46) in first jaw (42) as mainly recesses; with serrations (48) in second jaw (44) as mainly protrusions. Of course, serrations (46, 48) may take any other suitable form or may be simply omitted altogether. It should also be understood that serrations (46, 48) may be formed of an electrically non-conductive, or insulative, material, such as plastic, glass, and/or ceramic, for example, and may include a treatment such as polytetrafluoroethylene, a lubricant, or some other treatment to substantially prevent tissue from getting stuck to jaws (42, 44).

With jaws (42, 44) in a closed position, shaft (30) and end effector (40) are sized and configured to fit through trocars having various inner diameters, such that electrosurgical instrument (10) is usable in minimally invasive surgery, though of course electrosurgical instrument (10) could also be used in open procedures if desired. By way of example only, with jaws (42, 44) in a closed position, shaft (30) and end effector (40) may present an outer diameter of approximately 5 mm. Alternatively, shaft (30) and end effector (40) may present any other suitable outer diameter (e.g., between approximately 2 mm and approximately 20 mm, etc.).

As another merely illustrative variation, either jaw (42, 44) or both of jaws (42, 44) may include at least one port, passageway, conduit, and/or other feature that is operable to draw steam, smoke, and/or other gases/vapors/etc. from the surgical site. Such a feature may be in communication with a source of suction, such as an external source or a source within handpiece (20), etc. In addition, end effector (40) may include one or more tissue cooling features (not shown) that reduce the degree or extent of thermal spread caused by end effector (40) on adjacent tissue when electrode surfaces (50, 52) are activated. Various suitable forms that such cooling features may take will be apparent to those of ordinary skill in the art in view of the teachings herein.

In some versions, end effector (40) includes one or more sensors (not shown) that are configured to sense a variety of parameters at end effector (40), including but not limited to temperature of adjacent tissue, electrical resistance or impedance of adjacent tissue, voltage across adjacent tissue, forces exerted on jaws (42, 44) by adjacent tissue, etc. By way of example only, end effector (40) may include one or more positive temperature coefficient (PTC) thermistor bodies (54, 56) (e.g., PTC polymer, etc.), located adjacent to electrodes (50, 52) and/or elsewhere. Data from sensors may be communicated to controller (82). Controller (82) may process such data in a variety of ways. By way of example only, controller (82) may modulate or otherwise change the RF energy being delivered to electrode surfaces (50, 52), based at least in part on data acquired from one or more sensors at end effector (40). In addition or in the alternative, controller (82) may alert the user to one or more conditions via an audio and/or visual feedback device (e.g., speaker, lights, display screen, etc.), based at least in part on data acquired from one or more sensors at end effector (40). It should also be understood that some kinds of sensors need not necessarily be in communication with controller (82), and may simply provide a purely localized effect at end effector (40). For instance, a PTC thermistor bodies (54, 56) at end effector (40) may automatically reduce the energy delivery at electrode surfaces (50, 52) as the temperature of the tissue and/or end effector (40) increases, thereby reducing the likelihood of overheating. In some such versions, a PTC thermistor element is in series with power source (80) and electrode surface (50, 52); and the PTC thermistor provides an increased impedance (reducing flow of current) in response to temperatures exceeding a threshold. Furthermore, it should be understood that electrode surfaces (50, 52) may be used as sensors (e.g., to sense tissue impedance, etc.). Various kinds of sensors that may be incorporated into electrosurgical instrument (10) will be apparent to those of ordinary skill in the art in view of the teachings herein. Similarly various things that can be done with data from sensors, by controller (82) or otherwise, will be apparent to those of ordinary skill in the art in view of the teachings herein. Other suitable variations for end effector (40) will also be apparent to those of ordinary skill in the art in view of the teachings herein.

C. Exemplary Firing Beam

As also seen in FIGS. 2-4, electrosurgical instrument (10) of the present example includes a firing beam (60) that is longitudinally movable along part of the length of end effector (40). Firing beam (60) is coaxially positioned within shaft (30), extends along the length of shaft (30), and translates longitudinally within shaft (30) (including articulation section (36) in the present example), though it should be understood that firing beam (60) and shaft (30) may have any other suitable relationship. Firing beam (60) includes a sharp distal blade (64), an upper flange (62), and a lower flange (66). As best seen in FIG. 4, distal blade (64) extends through slots (46, 48) of jaws (42, 44), with upper flange (62) being located above jaw (44) in recess (59) and lower flange (66) being located below jaw (42) in recess (58). The configuration of distal blade (64) and flanges (62, 66) provides an “I-beam” type of cross section at the distal end of firing beam (60). While flanges (62, 66) extend longitudinally only along a small portion of the length of firing beam (60) in the present example, it should be understood that flanges (62, 66) may extend longitudinally along any suitable length of firing beam (60). In addition, while flanges (62, 66) are positioned along the exterior of jaws (42, 44), flanges (62, 66) may alternatively be disposed in corresponding slots formed within jaws (42, 44). For instance, each jaw (42, 44) may define a “T”-shaped slot, with parts of distal blade (64) being disposed in one vertical portion of each “T”-shaped slot and with flanges (62, 66) being disposed in the horizontal portions of the “T”-shaped slots. Various other suitable configurations and relationships will be apparent to those of ordinary skill in the art in view of the teachings herein.

Distal blade (64) is substantially sharp, such that distal blade (64) will readily sever tissue that is captured between jaws (42, 44). Distal blade (64) is also electrically grounded in the present example, providing a return path for RF energy as described elsewhere herein. In some other versions, distal blade (64) serves as an active electrode. In addition or in the alternative, distal blade (64) may be selectively energized with ultrasonic energy (e.g., harmonic vibrations at approximately 55.5 kHz, etc.).

The “I-beam” type of configuration of firing beam (60) provides closure of jaws (42, 44) as firing beam (60) is advanced distally. In particular, flange (62) urges jaw (44) pivotally toward jaw (42) as firing beam (60) is advanced from a proximal position (FIGS. 1-3) to a distal position (FIG. 4), by bearing against recess (59) formed in jaw (44). This closing effect on jaws (42, 44) by firing beam (60) may occur before distal blade (64) reaches tissue captured between jaws (42, 44). Such staging of encounters by firing beam (60) may reduce the force required to squeeze grip (24) to actuate firing beam (60) through a full firing stroke. In other words, in some such versions, firing beam (60) may have already overcome an initial resistance required to substantially close jaws (42, 44) on tissue before encountering resistance from severing the tissue captured between jaws (42, 44). Of course, any other suitable staging may be provided.

In the present example, flange (62) is configured to cam against a ramp feature at the proximal end of jaw (44) to open jaw (44) when firing beam (60) is retracted to a proximal position and to hold jaw (44) open when firing beam (60) remains at the proximal position. This camming capability may facilitate use of end effector (40) to separate layers of tissue, to perform blunt dissections, etc., by forcing jaws (42, 44) apart from a closed position. In some other versions, jaws (42, 44) are resiliently biased to an open position by a spring or other type of resilient feature. While jaws (42, 44) close or open as firing beam (60) is translated in the present example, it should be understood that other versions may provide independent movement of jaws (42, 44) and firing beam (60). By way of example only, one or more cables, rods, beams, or other features may extend through shaft (30) to selectively actuate jaws (42, 44) independently of firing beam (60). Such jaw (42, 44) actuation features may be separately controlled by a dedicated feature of handpiece (20). Alternatively, such jaw actuation features may be controlled by trigger (24) in addition to having trigger (24) control firing beam (60). It should also be understood that firing beam (60) may be resiliently biased to a proximal position, such that firing beam (60) retracts proximally when a user relaxes their grip on trigger (24).

D. Exemplary Operation

In an exemplary use, end effector (40) is inserted into a patient via a trocar. Articulation section (36) is substantially straight when end effector (40) and part of shaft (30) are inserted through the trocar. Articulation control (28) may then be manipulated to pivot or flex articulation section (36) of shaft (30) in order to position end effector (40) at a desired position and orientation relative to an anatomical structure within the patient. Two layers of tissue of the anatomical structure are then captured between jaws (42, 44) by squeezing trigger (24) toward pistol grip (22). Such layers of tissue may be part of the same natural lumen defining anatomical structure (e.g., blood vessel, portion of gastrointestinal tract, portion of reproductive system, etc.) in a patient. For instance, one tissue layer may comprise the top portion of a blood vessel while the other tissue layer may comprise the bottom portion of the blood vessel, along the same region of length of the blood vessel (e.g., such that the fluid path through the blood vessel before use of electrosurgical instrument (10) is perpendicular to the longitudinal axis defined by end effector (40), etc.). In other words, the lengths of jaws (42, 44) may be oriented perpendicular to (or at least generally transverse to) the length of the blood vessel. As noted above, flanges (62, 66) cammingly act to pivot jaw (42) toward jaw (44) when firing beam (60) is actuated distally by squeezing trigger (24) toward pistol grip (22). Jaws (42, 44) may be substantially clamping tissue before trigger (24) has swept through a full range of motion toward pistol grip (22), such that trigger (24) may continue pivoting toward pistol grip (22) through a subsequent range of motion after jaws (42, 44) have substantially clamped on the tissue.

With tissue layers captured between jaws (42, 44) firing beam (60) continues to advance distally by the user squeezing trigger (24) further toward pistol grip (22). As firing beam (60) continues to advance distally, distal blade (64) simultaneously severs the clamped tissue layers, resulting in separated upper layer portions being apposed with respective separated lower layer portions. In some versions, this results in a blood vessel being cut in a direction that is generally transverse to the length of the blood vessel. It should be understood that the presence of flanges (62, 66) immediately above and below jaws (42, 44), respectively, may help keep jaws (42, 44) in a closed and tightly clamping position. In particular, flanges (62, 66) may help maintain a significantly compressive force between jaws (42, 44). With severed tissue layer portions being compressed between jaws (42, 44), electrode surfaces (50, 52) are activated with bipolar RF energy by the user depressing activation button (26). In some versions, electrodes (50, 52) are selectively coupled with power source (80) (e.g., by the user depressing button (26), etc.) such that electrode surfaces (50, 52) of jaws (42, 44) are activated with a common first polarity while firing beam (60) is activated at a second polarity that is opposite to the first polarity. Thus, a bipolar RF current flows between firing beam (60) and electrode surfaces (50, 52) of jaws (42, 44), through the compressed regions of severed tissue layer portions. In some other versions, electrode surface (50) has one polarity while electrode surface (52) and firing beam (60) both have the other polarity. In either version (among at least some others), bipolar RF energy delivered by power source (80) ultimately thermally welds the tissue layer portions on one side of firing beam (60) together and the tissue layer portions on the other side of firing beam (60) together.

In certain circumstances, the heat generated by activated electrode surfaces (50, 52) can denature the collagen within the tissue layer portions and, in cooperation with clamping pressure provided by jaws (42, 44), the denatured collagen can form a seal within the tissue layer portions. Thus, the severed ends of the natural lumen defining anatomical structure are hemostatically sealed shut, such that the severed ends will not leak bodily fluids. In some versions, electrode surfaces (50, 52) may be activated with bipolar RF energy before firing beam (60) even begins to translate distally and thus before the tissue is even severed. For instance, such timing may be provided in versions where button (26) serves as a mechanical lockout relative to trigger (24) in addition to serving as a switch between power source (80) and electrode surfaces (50, 52). Other suitable ways in which instrument (10) may be operable and operated will be apparent to those of ordinary skill in the art in view of the teachings herein.

II. Exemplary End Effector with Staged Control of Outer Beam and Inner Beam

As described above, jaws (42, 44) clamp tissue, blade (64) cuts tissue, and electrode surfaces (50, 52) energize tissue in order to seal tissue. As also described above, a single actuation of firing beam (60) essentially causes the abovementioned clamping and cutting to occur substantially simultaneously. In some instances, it may be desirable to control the aforementioned actions in separate stages. For instance, the user may wish to clamp or close jaws (42, 44) around tissue prior to cutting or sealing tissue. Pausing between the clamping, cutting, and sealing of the tissue may enable the user to more closely examine the surgical area between each step. Thereafter, when the user is ready, the tissue may be cut and sealed according to when the user wishes to cut and seal the tissue. It will also be appreciated that by separating the actuations of clamping/closing, cutting, and sealing, the user may even manually apply intermediate steps in between the clamping, cutting, and sealing. For instance, after clamping the tissue, the user could manipulate end effector (40) with tissue clamped between jaws (42, 44) such that the user can better view the surgical area. The user may even attempt to re-clamp the area of tissue since the tissue has not yet been cut or sealed. Thereafter, the user may initiate cutting and sealing. In some instances, the user may wish to compress an area of tissue with relatively light compression followed by using a higher compressive force. In other instances, the user may wish to use either light compressive force or high compressive force. In yet other instances, the user may wish to grasp, compress, and seal the relevant tissue area before cutting and/or transecting tissue. Other uses of separately controlling clamping, cutting, and sealing will be apparent to one of ordinary skill in the art in view of the teachings herein.

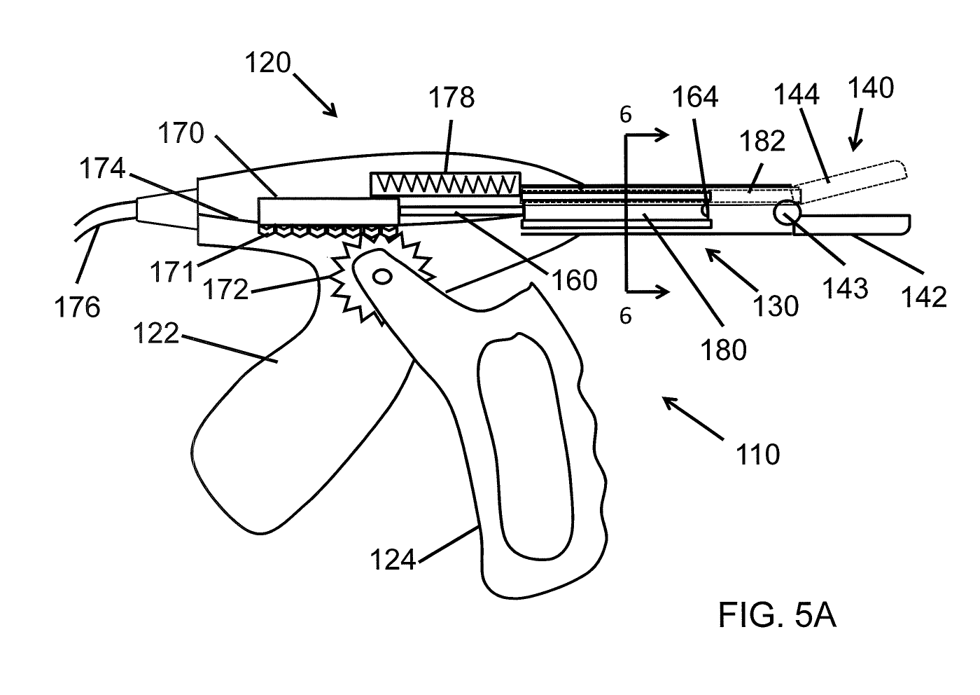

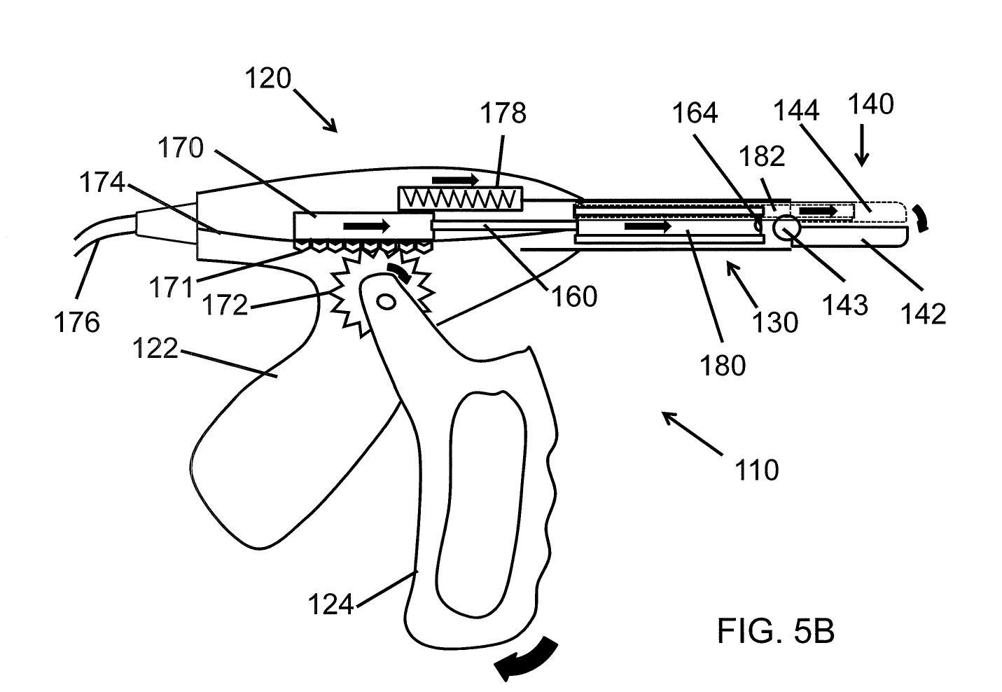

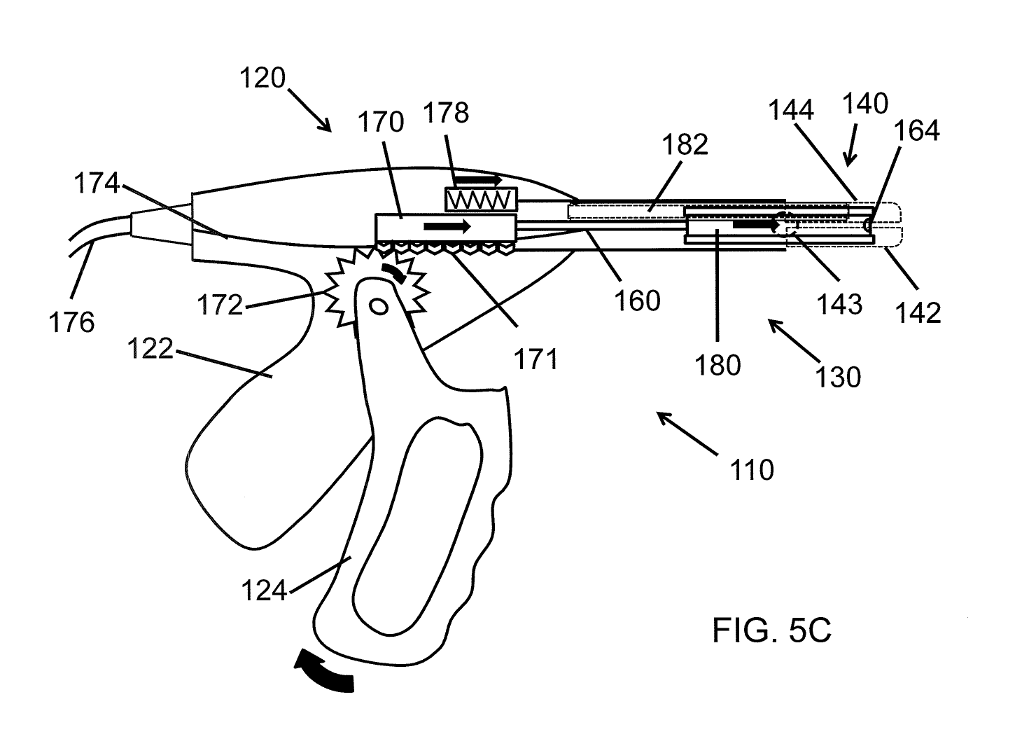

FIG. 5A shows an exemplary version of an electrosurgical instrument (110) comprising a handpiece (120), a shaft (130), and an end effector (140). Specific details with respect to the operation of electrosurgical instrument (110) will be discussed in further detail below. In general, instrument (110) is operable to clamp tissue, cut tissue, and seal tissue in a manner that allows the user to separately control the separate actions of clamping, cutting, and sealing.

Handpiece (120) comprises a pistol grip (122) and a pivoting trigger (124). Handpiece (120) further comprises a control rack (170) and pinion (172). Pinion (172) is in communication with pivoting trigger (124). Handpiece (120) further comprises wiring (174), spring feature (178), and firing beam (160). The various components within handpiece (120) will be described in further detail below. Firing beam (160) of handpiece (120) is in communication with inner beam (180). Inner beam (180) is in communication with outer beam (182), and both inner beam (180) and outer beam (182) are positioned within shaft (130). Inner beam (180) comprises a blade (164) at the distal end of inner beam (180). Shaft (130) in the exemplary version is unitarily coupled to first jaw (142) and second jaw (144) is pivotally coupled to first jaw (142) through pivotal coupling (143).

Pistol grip (122) of the exemplary version shown in FIG. 5A is operable to be gripped using a single hand such that a user can grasp pistol grip (122) and simultaneously actuate pivoting trigger (124). Pivoting trigger (124) is operable to pivot in relation to pistol grip (122). It will be appreciated that pivoting trigger (124) and pistol grip (122) of the exemplary version are substantially similar to pivoting trigger (24) and pistol grip (22) of FIG. 1 with the exception that the overall shape of pivoting trigger (124) and pistol grip (122) differs from pivoting trigger (24) and pistol grip (22). However, it will be understood that the shape of pivoting trigger (124) and pistol grip (122) could have the same or substantially similar shape as pivoting trigger (24) and pistol grip (22) shown in FIG. 1.

Pivoting trigger (124) pivots with pinion (172) about a pin. Pinion (172) engages teeth (171) of control rack (170). While pinion (172) and teeth (171) are used in the illustrated version, it will be understood that any suitable mechanism for converting rotational motion into linear motion may be used as would be apparent to one of ordinary skill in the art in view of the teachings herein. Teeth (171) comprise a plurality of linearly arranged teeth operable to engage pinion (172) such that when pinion (172) rotates clockwise in FIG. 5A, control rack (170) advances, whereas when pinion (172) rotates counter-clockwise, control rack (170) retracts.

Control rack (170) is in communication with one end of spring (178). The opposite end of spring (178) is in communication with shaft (130), or any other suitable longitudinally stationary structure within instrument (110). Spring (178) is operably biased to remain in the position shown in FIG. 5A, such that spring (178) biases control rack (170) to the proximal position. Furthermore, spring (178) is positioned substantially parallel to the longitudinal axis of control rack (170) and firing beam (160). Thus, as spring (178) contracts, spring (178) is biased to expand back to the position in FIG. 5A. Furthermore, if spring (178) expands beyond the position shown in FIG. 5A, then spring (178) retracts to return to the position shown in FIG. 5A. While the exemplary version uses spring (178), it will be understood that any suitable structure for longitudinally biasing control rack (170) to the position shown in FIG. 5A may be used as would be apparent to one of ordinary skill in the art in view of the teachings herein.

Wiring (174) is in communication with cable (176) and end effector (140) such that energy may be delivered to jaws (142, 144) from cable (176) via wiring (174). Cable (176) is in communication with a power source and controller similar to power source (80) and controller (82) as shown in FIG. 2. However, it will be understood that cable (176) may be in communication with any suitable structure operable to deliver RF energy as would be apparent to one of ordinary skill in the art in view of the teachings herein. Furthermore, while in the exemplary version, cable (176) suggests that the power source and controller be positioned externally from handpiece (120), it will be appreciated that the power source and controller may instead be positioned within handpiece (120). For instance, in the event that the power source comprises a battery, then the power source and controller may be positioned within handpiece (120). Other suitable configurations will be apparent to one of ordinary skill in the art in view of the teachings herein. Wiring (174) is operable to deliver bipolar energy to jaw (142, 144). For instance, wiring (174) may comprise a plurality of wires such that at least one wire serves as an active wire to jaw (142) while another wire serves as a ground return path for jaw (144), thereby providing jaws (142, 144) with bipolar energy for welding a tissue site with RF energy. Jaws (142, 144) of end effector (140) include electrode surfaces similar to electrode surfaces (50, 52) shown in FIG. 3 operable to deliver RF energy to tissue to seal tissue.

Firing beam (160) is operable to move longitudinally along shaft (130). Firing beam (160) is in communication with inner beam (180) such that as firing beam (160) advances, inner beam (180) also advances. While in the exemplary version, firing beam (160) and inner beam (180) appear to be two separate components joined together, it will be appreciated that firing beam (160) and inner beam (180) may be unitarily constructed. For instance, firing beam (160) may be constructed substantially similarly to firing beam (60) shown in FIG. 2.

Inner beam (180) is surrounded by outer beam (182), which will be discussed in further detail below. Outer beam (182) extends longitudinally through shaft (130). Outer beam (182) has a length longer than inner beam (180) such that as inner beam (180) and outer beam (182) advance together along shaft (130), outer beam (182) advances ahead of inner beam (180). Once outer beam (182) reaches a distal-most position, inner beam (180) continues to advance distally relative to shaft (130) and relative to outer beam (182) until inner beam (180) reaches a distal-most position.

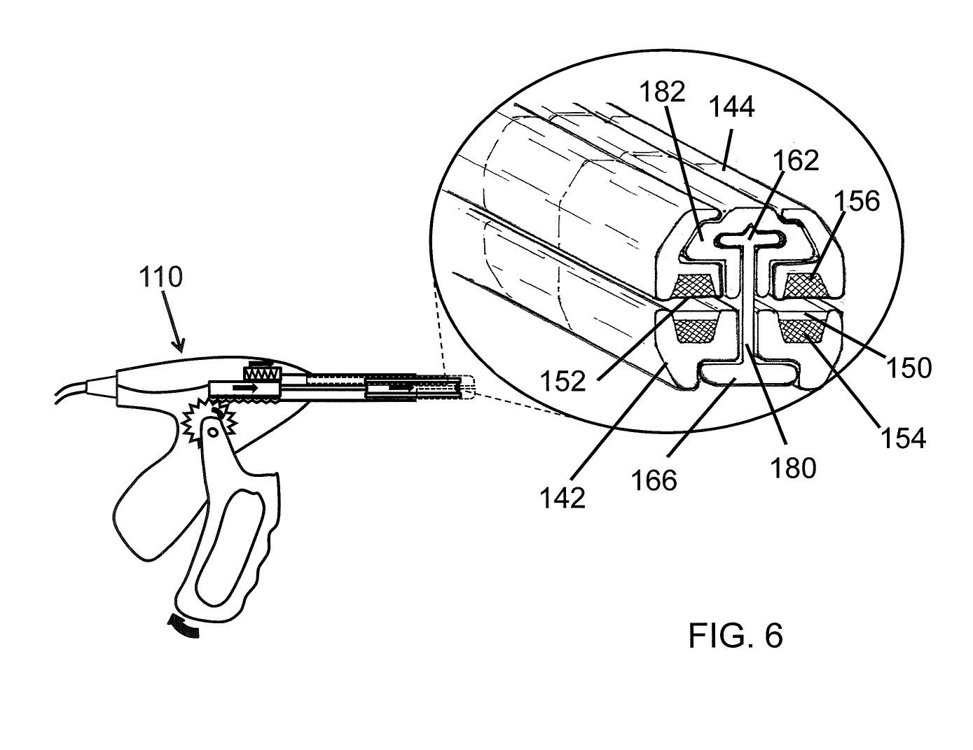

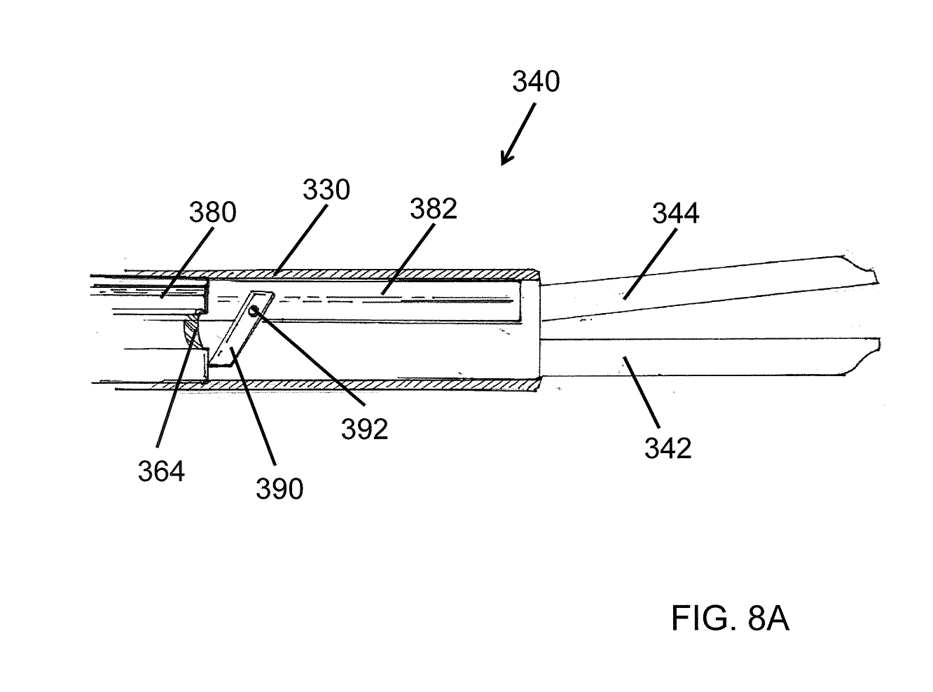

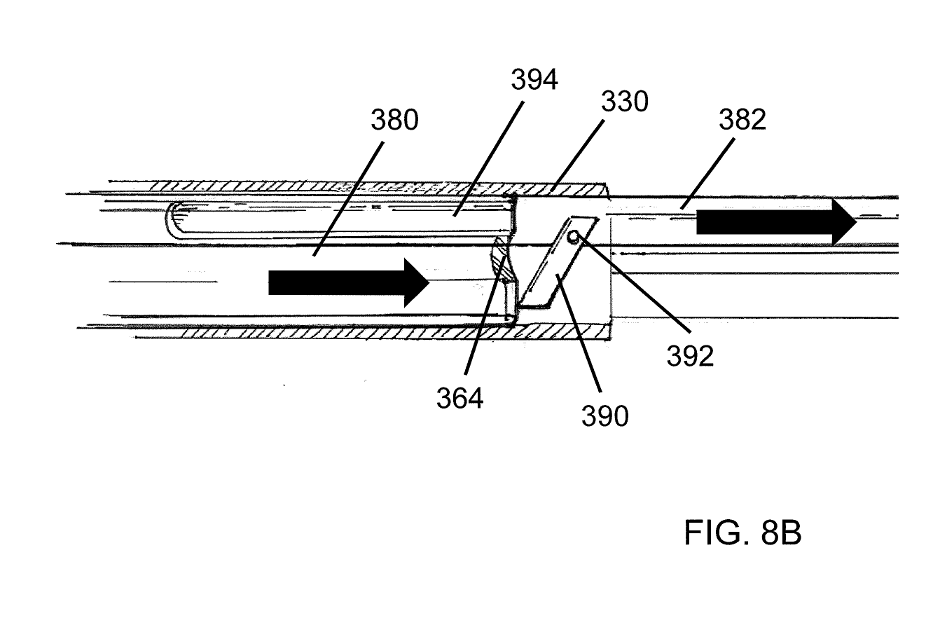

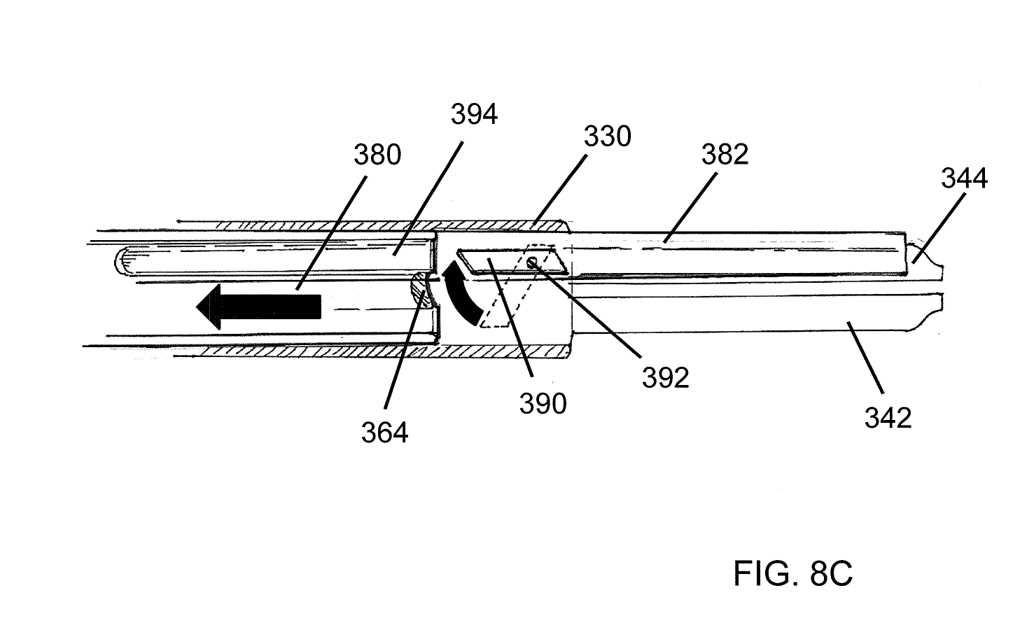

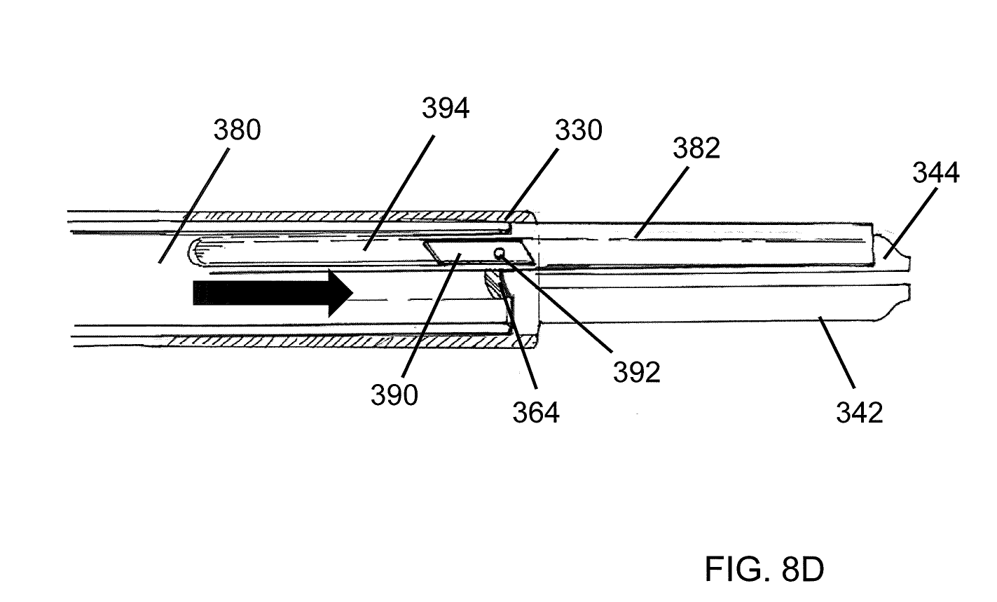

FIG. 6 shows a cross sectional view of inner beam (180). Inner beam (180) comprises an upper flange (162) and a lower flange (166). The upper portion of inner beam (180) fits within outer beam (182). Outer beam (182) is shaped to complement the shape of inner beam (180). In particular, in the exemplary version, inner beam (180) has a cross section shaped similar to an I-beam, whereas outer beam (182) has a cross section shaped similar to a sideways C to complement inner beam (180). However, it will be appreciated that inner beam (180) and outer beam (182) may have any suitable shape operable to complement each other. Outer beam (182) is shaped further to fit within jaw (144). Inner beam (180) and lower flange (166) are operable to fit within jaw (142). Thus, as outer beam (182) and inner beam (180) are advanced fully within jaws (142, 144), jaws (142, 144) remain shut. Furthermore, as outer beam (182) translates longitudinally within jaw (144), jaw (144) closes down upon jaw (142), which will be discussed in further detail below. Outer beam (182) and inner beam (180) are operable to advance within shaft (130) in response to firing beam (160) being advanced within shaft (130). Furthermore, outer beam (182) and inner beam (180) are able to selectively engage and disengage such that firing beam (160) initially advances to advance both inner beam (180) and outer beam (182). Thereafter, outer beam (182) and inner beam (180) may be disengaged such that inner beam (180) may be advanced independently of outer beam (182). More specific details regarding engaging and disengaging inner beam (180) and outer beam (182) will be described below when FIGS. 8A-11C are discussed.

As also seen in FIG. 6, jaws (142, 144) comprise electrode surfaces (150, 152) and thermistor bodies (154, 156). It will be appreciated that electrode surfaces (150, 152) and thermistor bodies (154, 156) are substantially similar to electrode surfaces (50, 52) and thermistor bodies (54, 56) shown in FIG. 4. However, it will be understood that other suitable structures may be used to deliver RF energy to tissue and to dissipate thermal energy accordingly as would be apparent to one of ordinary skill in the art in view of the teachings herein.

FIG. 5A shows instrument (110) as it is being prepared to fire. The user may grasp pistol grip (122) and actuate pivoting trigger (124). Once the user positions end effector (140) near an appropriate portion of tissue and is ready to clamp the tissue, the user may actuate pivoting trigger (124) moving it approximately to the position shown in FIG. 5B. When pivoting trigger (124) actuates, pinion (172) rotates causing control rack (170) to advance as pinion (172) engages control rack (170) through teeth (171). Firing beam (160) advances, which advances inner beam (180). Since inner beam (180) is engaged with outer beam (182), outer beam (182) also advances such that outer beam (182) moves through jaw (144), thereby closing jaw (144) toward jaw (142). Spring (178) compresses as control rack (170) advances, thereby applying a slight proximal bias to control rack (170). In other exemplary versions, pinion (172) may be equipped with a ratcheting feature such that despite any proximal bias applied by spring (178), control rack (170) maintains its longitudinal position. Such a ratcheting feature may include a manual release controlled through a separate input (e.g. button, switch, etc.) on handpiece (120).

After reaching the position shown in FIG. 5B, inner beam (180) and outer beam (182) may be disengaged in any suitable manner. For instance, any of implements shown in FIGS. 8A-11C may be used as will be discussed in further detail below. After disengaging inner beam (180) from outer beam (182), pivoting trigger (124) may be further actuated as shown in FIG. 5C. Pinion (172) rotates further, thereby advancing control rack (170) further. Firing beam (160) advances further thereby advancing inner beam (180) while outer beam (182) remains stationary. Inner beam (180) advances through jaws (142, 144), thereby cutting tissue between jaws (142, 144) with blade (164). As inner beam (180) advances to cut tissue, it will be appreciated that inner beam (180) further secures the closure of jaws (142, 144). Spring (178) further compresses, thereby causing a stronger proximal bias of control rack (170) than the bias shown in FIG. 5B. As the user actuates pivoting trigger (124) to move to the position shown in FIG. 5C, a control such as control (82) shown in FIG. 2 may be engaged to energize electrode surfaces (150, 152) to seal tissue sandwiched between jaws (142, 144). It will be appreciated that sealing tissue may also occur prior to or even after actuating pivoting trigger (124) as seen in FIG. 5C.

Once tissue has been cut and sealed, the user may release pivoting trigger (124). Due to bias stored in spring (178), spring (178) then retracts control rack (170), which rotates pinion (172) to return pivoting trigger (124) to the position shown in FIG. 5A. Additionally, as control rack (170) retracts, firing beam (160) retracts, which retracts inner beam (180). Inner beam (180) then re-engages outer beam (182), which will be discussed in further detail below, and subsequently retracts outer beam (182). As a result, jaws (142, 144) release tissue and allow the user to remove instrument (110) from the surgical site.

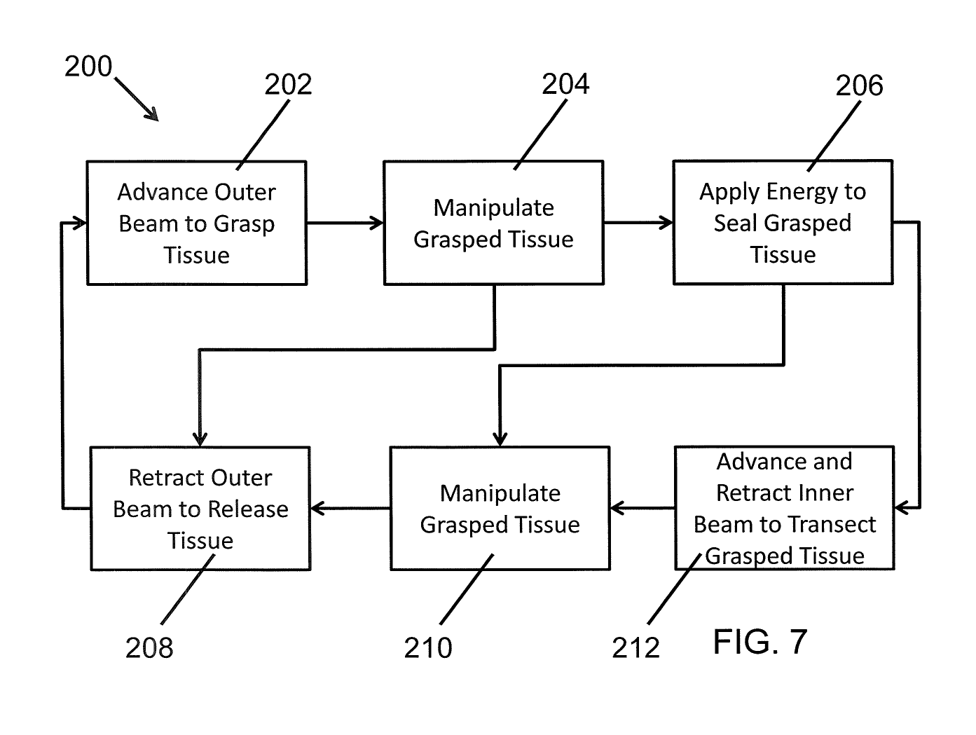

It will be understood that a variety of different ways of using instrument (110) may be used. For instance, the above mentioned way of using instrument (110) represents only one possible way that a user may use instrument (110) to close, cut, and seal tissue. FIG. 7 shows steps that may be used for instrument (110) in a less linear manner than described above. It will be appreciated that controlling inner beam (180) and outer beam (182) in separate stages enables the user to choose the appropriate steps for the procedure being used. In general, a flowchart (200) may be used to guide the actions of a user using instrument (110). In step (202), the user may advance outer beam (182) to grasp tissue. In particular, advancing outer beam (182) closes jaw (144) towards jaw (142) to close jaws (142, 144) around tissue. In step (204), the user may manipulate grasped tissue. The user may do so for a variety of reasons. For instance, the user may wish to view a different portion of the tissue that is only visible by manipulating the grasped tissue. Furthermore, there may be therapeutic value in manipulating the grasped tissue prior to cutting or sealing. A cleaner seal or cut may be attainable by manipulating the tissue or readjusting the position of instrument (110) prior to sealing or cutting.

After step (204), the user may perform step (206) or step (208). In the event that tissue needs to be re-grasped or simply released, in step (208), the user may retract outer beam (182), thereby releasing the tissue from jaws (142, 144). Alternatively, if the user wishes to energize the tissue to seal it, step (206) may be performed which applies energy to seal grasped tissue via electrode surfaces (150, 152). Thereafter, the user may perform step (210), which manipulates the grasped tissue similarly to step (204). Alternatively, the user may perform step (212), which advances inner beam (180) to transect grasped tissue. Thereafter, the user may manipulate grasped tissue in step (210). The user may then retract outer beam (182) to release grasped tissue. The user may be completed with the procedure or may return to step (202) to repeat the procedure by grasping another portion of tissue. Other suitable ways in which instrument (110) may be used will be apparent to one of ordinary skill in the art in view of the teachings herein.

A. Exemplary Pawl Disengaging Feature

As was discussed earlier, there may be various ways of disengaging inner beam (180) and outer beam (182), thereby allowing inner beam (180) to advance independently of outer beam (182). FIGS. 8A-8D show one exemplary feature that may be used for disengaging and engaging inner beam (180) and outer beam (182).

FIG. 8A shows an exemplary end effector (340) similar to end effector (140) of FIG. 5A. The primary difference between end effector (340) and end effector (140) being the end effector (340) includes a pawl feature (390). End effector (340) comprises a first jaw (342) and second jaw (344) that extend from a shaft (330). Jaws (342, 344) are substantially similar to jaws (142, 144). An inner beam (380) and outer beam (382) extend through shaft (330). Inner beam (380) comprises a blade (364) at the distal edge of inner beam (380). Blade (364) and shaft (330) are substantially similar to blade (164) and shaft (130). Outer beam (382) of the exemplary version comprises pawl feature (390) which is connected to outer beam (382) through a pawl pivot (392). Pawl feature (390) has a parallelogram shape, but it will be appreciated that any suitable shape may be used for pawl feature (390). For instance, an elliptical, peg, straight rectangle, trapezoidal, or any other suitable shape may be used. Pawl feature (390) engages the lower flange of inner beam (380) when pawl feature (390) is pivoted downward, thereby coupling the motion of inner beam (380) and outer beam (382). As a result, when inner beam (380) advances as shown in FIG. 8B, outer beam (382) also advances. It will be appreciated that pawl feature (390) may be used in conjunction with the version shown in FIG. 5B such that as inner beam (180) advances in response to actuating pivoting trigger (124), outer beam (182) also advances.

After outer beam (382) has advanced sufficiently along jaw (344), inner beam (380) is retracted by the user as shown in FIG. 8C while outer beam (382) remains longitudinally stationary. Pawl pivot (392) is spring biased such that when pawl feature (390) is not engaged with inner beam (380), pawl pivot (392) is biased to rotate upwards as shown in FIG. 8C. Thus, as the user retracts inner beam (380), pawl feature (390) rotates upward and remains in a position generally parallel to outer beam (382). Inner beam (380) has a pawl pocket (394) formed within inner beam (380) shaped generally to allow pawl feature (390) to fit within pawl pocket (394). Furthermore, pawl pocket (394) is shaped to be deep enough such that pawl feature (390) can fit deeply within pawl pocket (394), thereby allowing inner beam (380) to advance along jaws (342, 344) without being hindered by pawl feature (390).

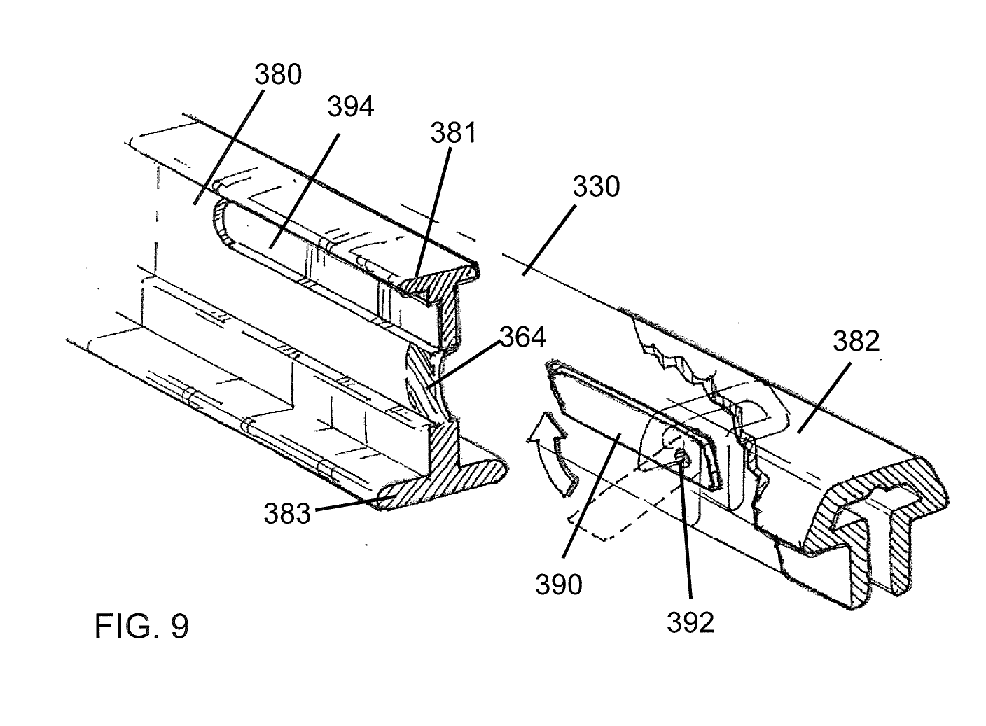

FIG. 8D shows inner beam (380) advanced such that pawl feature (390) is positioned within pawl pocket (394). Pawl pocket (394) can be seen more clearly in FIG. 9, which shows pawl pocket (394) as forming a recess within inner beam (380). A firing beam such as firing beam (160) shown in FIG. 5A may be advanced to advance inner beam (380) along shaft (330). As can be seen in FIG. 8D, pawl pocket (394) has enough depth clearance such that inner beam (380) can advance further along shaft until inner beam (380) advances along jaws (342, 344), thereby allowing blade (364) to cut tissue closed between jaws (342, 344). Pawl feature (390) simply nests further within pawl pocket (394) as inner beam (380) advances. Jaws (342, 344) may also include sealing features such as electrode surfaces (150, 152), which were shown in FIG. 6, to enable jaws (342, 344) to seal tissue before, during, and/or after the tissue is cut. After the tissue is clamped, cut, and sealed, the user may then retract inner beam (380). A spring such as spring (178) shown in FIG. 5A may be used to enable inner beam (380) to retract by applying a proximal bias to firing rod (160), which retracts inner beam (380). Furthermore, inner beam (380) may have a detent or other catching feature such that as inner beam (380) retracts to approximately the position of inner beam (380) shown in FIG. 8C, inner beam (380) couples with outer beam (382), thereby causing outer beam (382) to also retract as inner beam (380) is retracted further. As a result, both inner beam (380) and outer beam (382) retracts approximately to the position shown in FIG. 8A, which allows jaws (342, 344) to release grasped tissue and further allows the user to remove end effector (340) from the surgical area. A feature such as a switch, lever, tab, or any other suitable feature positioned on shaft (330), jaw (344), and/or elsewhere may be used to pivot pawl feature (390) to the downward position shown in FIG. 8A as outer beam (382) is retracted.

B. Exemplary Detent Disengaging Feature

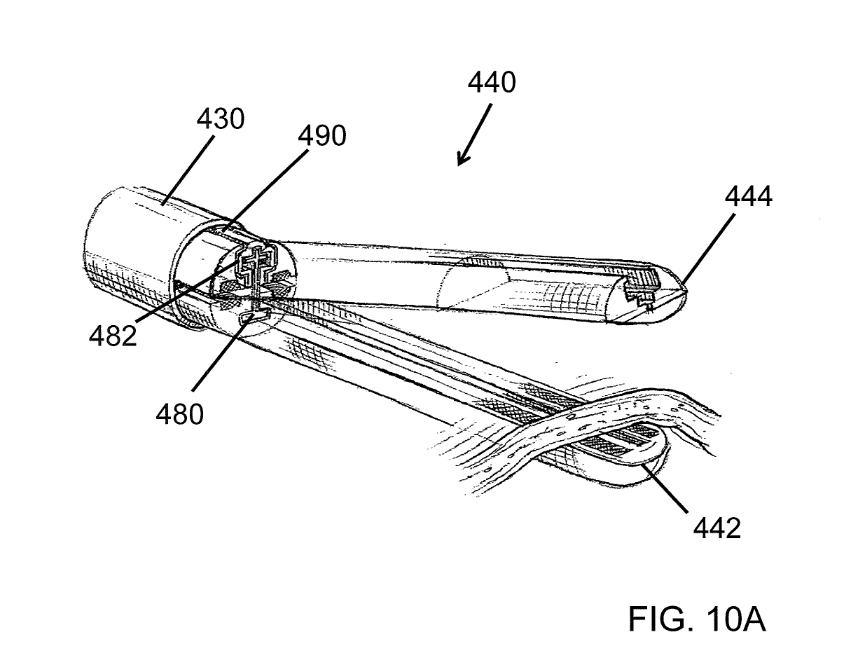

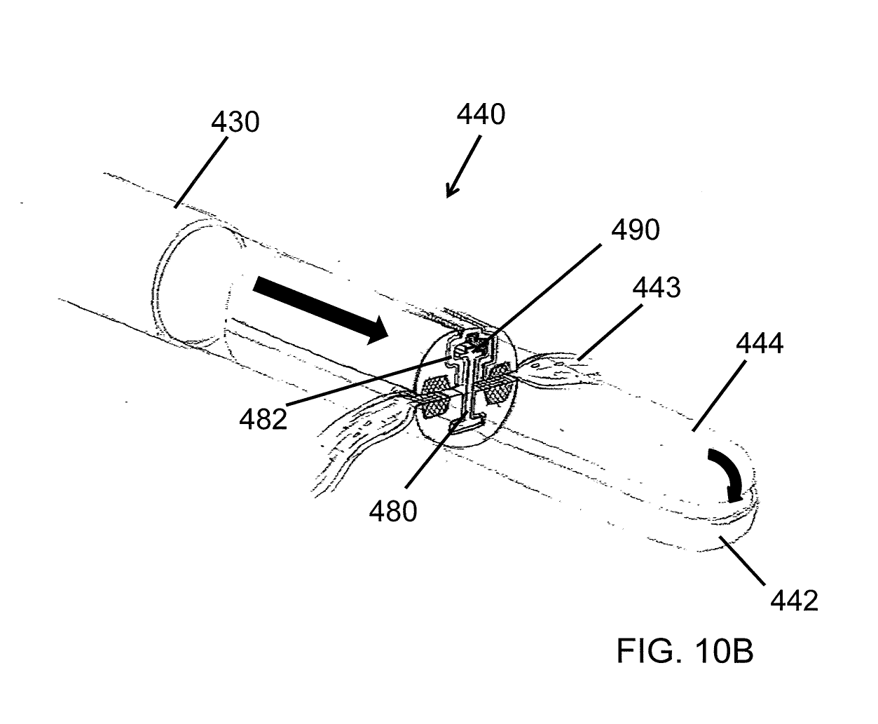

While FIGS. 8A-9 show one exemplary way of engaging and disengaging inner beam (380) from outer beam (382), FIG. 10A-12C show yet another exemplary mechanism for engaging and disengaging an inner beam and an outer beam such as inner beam (180) and outer beam (182) shown in FIG. 5A. FIG. 10A shows an exemplary end effector (440), which may be substantially similar to end effector (140) with the primary difference being that end effector (440) includes a detent feature (390), which will be discussed in further detail below. In fact, end effector (440) may simply be used in place of end effector (140) of FIG. 5A.

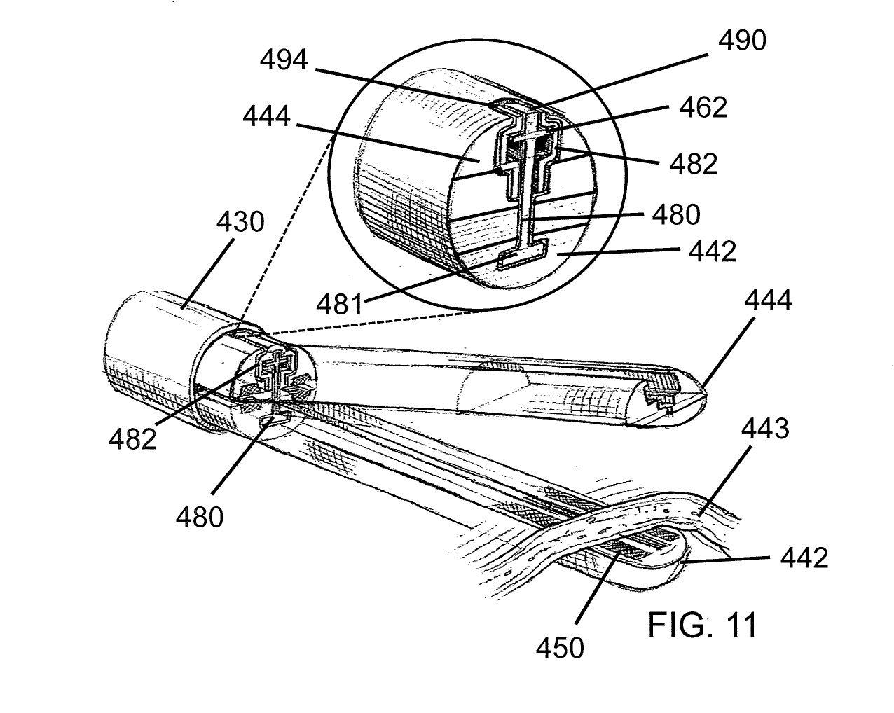

End effector (440) comprises a first jaw (442) and second jaw (444), which may be substantially similar to jaws (142, 144) shown in FIG. 5B. Jaw (442) comprises an electrode surface (450) with another electrode surface (452) on jaw (444) operable to seal tissue (443) similar to electrode surfaces (150, 152). End effector (440) further comprises a shaft (430) having an outer beam (482) and inner beam (480) extending through shaft (430). Inner beam (480) comprises an upward facing detent feature (490) operable to engage a detent pocket (494) formed within outer beam (482), which may be seen in FIG. 11. Inner beam (480) and outer beam (482) translate together relative to jaws (442, 444) when detent feature (490) is disposed in detent pocket (494). As inner beam (480) advances distally through jaws (442, 444), inner beam (480) eventually reaches a point where it drops slightly, moving toward jaw (442). As a result, detent feature (490) disengages detent pocket (494). It will be appreciated that outer beam (482) provides sufficient clearance such that upper flange (462) of inner beam (480) can lower or raise in relation to outer beam (482). When detent feature (490) disengages detent pocket (494), inner beam (480) may be advanced independently from outer beam (482).

FIG. 10B shows inner beam (480) and outer beam (482) advanced within shaft (430) such that jaw (444) closes upon jaw (442). It will be appreciated that advancing inner beam (480) and outer beam (482) may be accomplished similarly to advancing inner beam (180) and outer beam (182) shown in FIG. 5B by actuating a pivoting trigger (124) to advance firing beam (160) to advance inner beam (480). As outer beam (482) advances within jaw (444), jaw (444) closes upon tissue (443) for sealing and cutting. By advancing along shaft (430) and through jaws (442, 444), inner beam (480) descends within outer beam (482), thereby causing detent feature (490) to disengage detent pocket (492). The descent of inner beam (480) occurs as a result of a ramping feature, which will be discussed in further detail below.

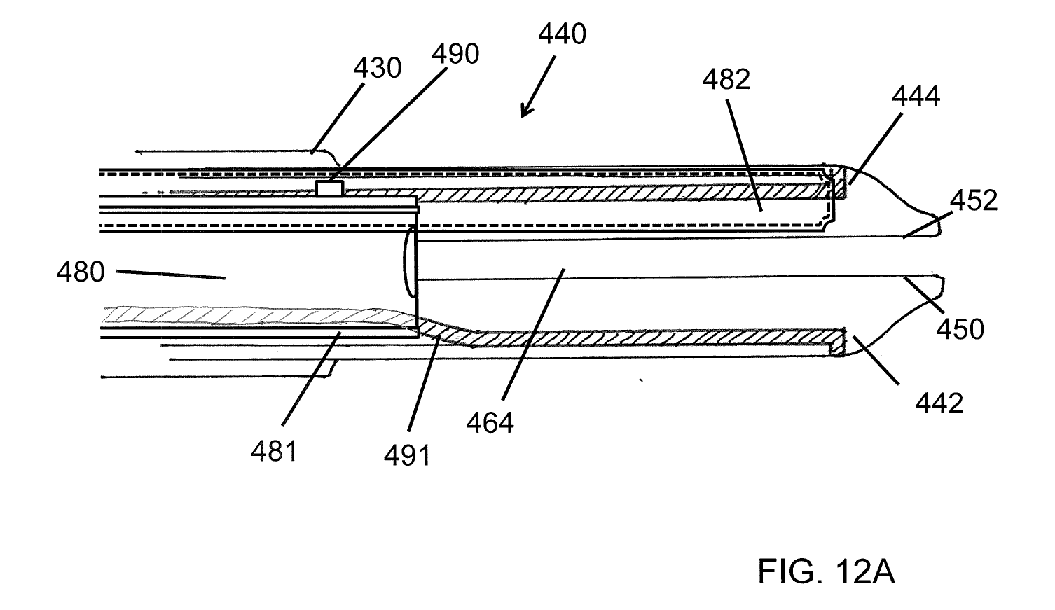

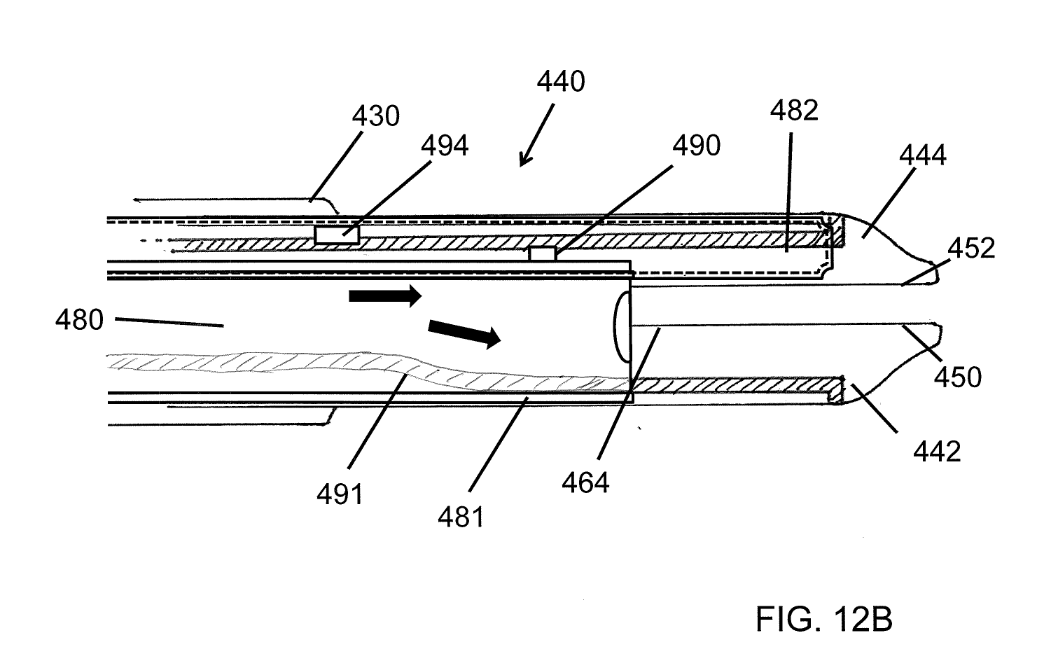

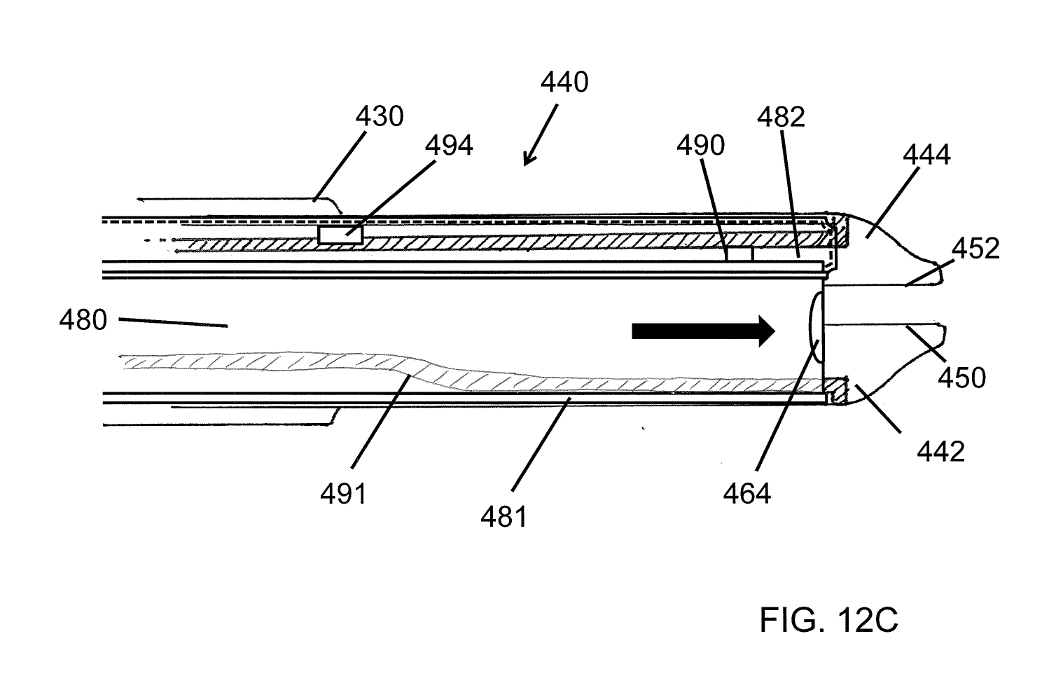

Once detent feature (490) disengages detent pocket (494), the user may actuate pivoting trigger (124) of FIG. 5A further thereby causing firing beam (160) to advance further and causing inner beam (480) to advance. Inner beam (480) comprises a blade (464) shown in FIG. 12A such that as inner beam (480) advances further, blade (464) cuts tissue (443). Prior to, during, or after cutting tissue (443), electrode surfaces (450, 452) may deliver RF energy to tissue (443), thereby sealing tissue (443).

FIG. 12A shows an exemplary cross sectional side view of end effector (440) such that ramp feature (491) is visible. Ramp feature (491) is defined by an exterior surface of jaw (442) adjacent to the slot of jaw (442) through which inner beam (480) advances. Ramp feature (491) presents a gently downward sloping ramp positioned such that a lower flange (481) of inner beam (480) rides below ramp feature (491). At the stage of operation shown in FIG. 12A, outer beam (482) is already advanced to a distal position such that outer beam (482) has closed jaw (444) against jaw (442). As inner beam (480) continues to advance through shaft (430) and jaws (442, 444), lower flange (481) rides down along the slope of ramp feature (491), thereby leading inner beam (480) to a vertically lower position, which can be seen in FIG. 12B. It should be understood that outer beam (482) is positioned to advance ahead of inner beam (480), such that outer beam (482) closes jaws before inner beam (480) starts to cut tissue (443) and before inner beam (480) starts to travel vertically downwardly. As also seen in FIG. 12B, detent feature (490) has disengaged detent pocket (494) such that inner beam (480) can advance independently of outer beam (482).

FIG. 12C shows inner beam (480) fully advanced such that blade (464) has transected any tissue positioned between jaws (442, 444). Similar to FIG. 12B, inner beam (480) remains in the vertically downward position at the stage shown in FIG. 12C. When the cutting and sealing of tissue (443) is complete, the user may then retract inner beam (480). It will be appreciated that retracting inner beam (480) may be performed manually by the user. In particular, the user may actuate a pivoting trigger (124) as seen in FIG. 5A such that firing beam (160) retracts, thereby retracting inner beam (480). Alternatively, a spring such as spring (178) may provide a proximal bias thereby also retracting firing beam (160). As inner beam (480) retracts, inner beam (480) ascends the ramp feature (491) thereby leading detent feature (490) to re-engage detent pocket (494). Upon re-engaging detent pocket (494) with detent feature (490), inner beam (480) and outer beam (482) become coupled such that as inner beam (480) retracts, outer beam (482) also retracts. As outer beam (482) retracts, outer beam (482) allows jaws (442, 444) to open, thereby releasing tissue (443).

III. Exemplary Firing Beam Driver

It will be understood that in some instances, in addition to providing independent control of closing, cutting, and sealing tissue, it may be desirable to provide additional force during the act of clamping/closing and cutting tissue. In particular, as tissue is placed between jaws such as jaws (142, 144), tissue may be thick enough such that jaws (142, 144) may not close completely. As a result, it may be desirable to provide increased closure force at jaws (142, 144) such that jaws (142, 144) may fully close upon tissue.

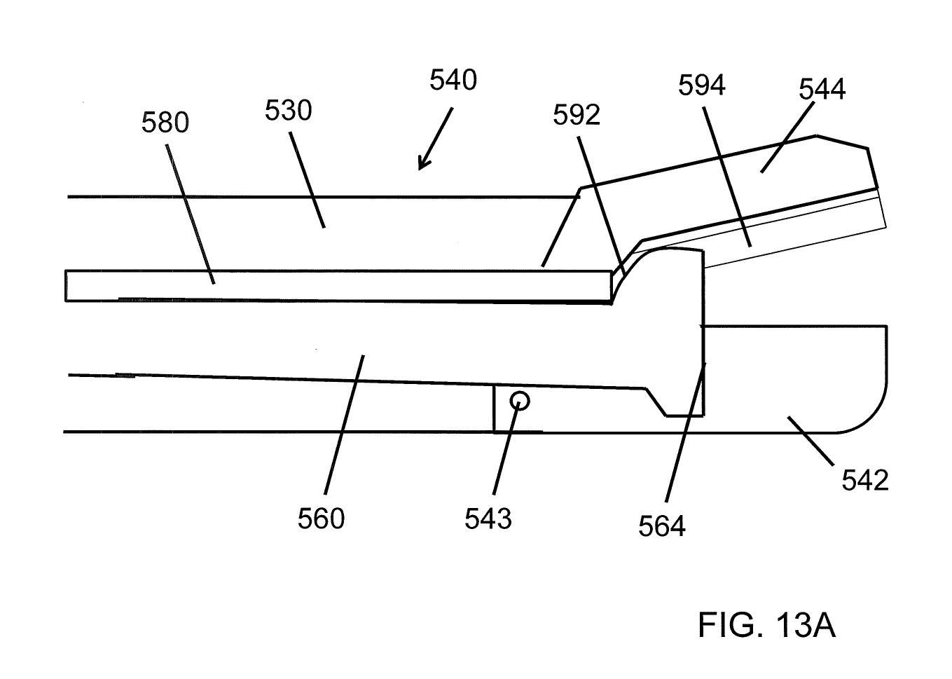

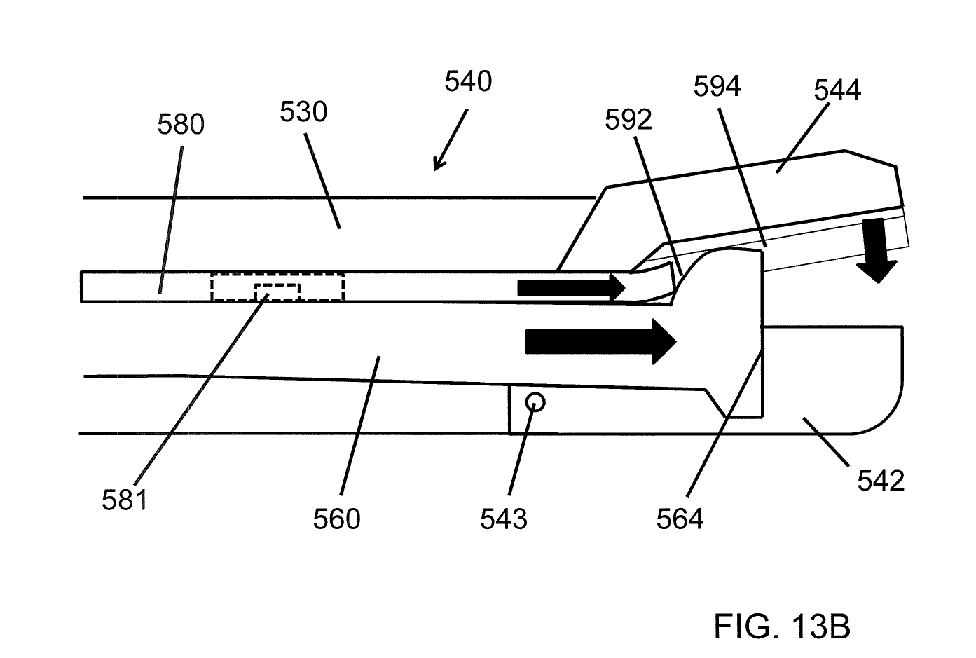

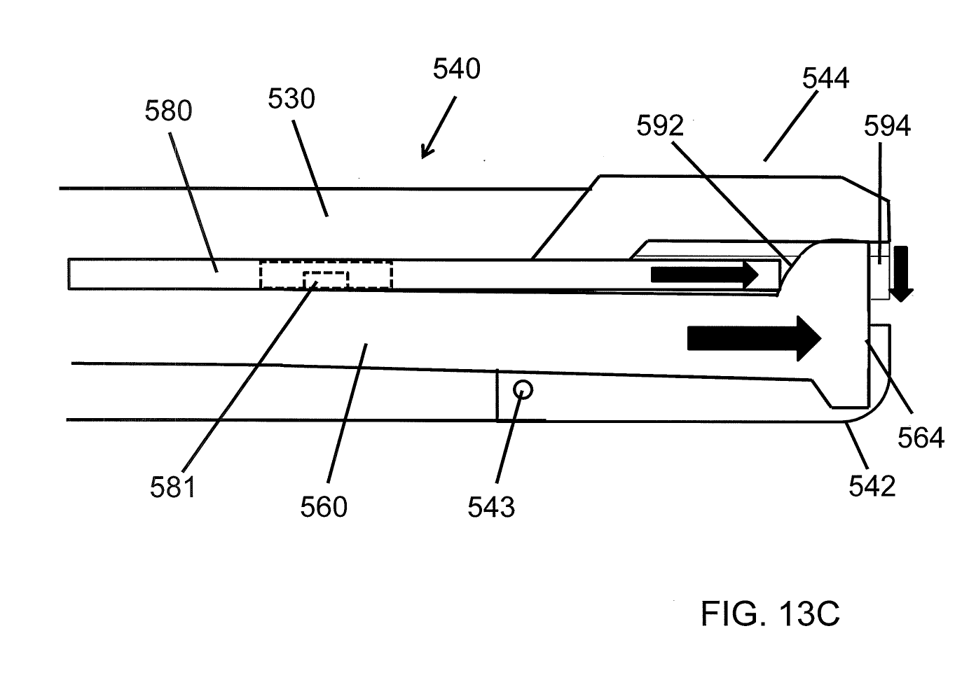

FIG. 13A depicts an exemplary end effector (540) having shaft (530) with a first jaw (542) and second jaw (544) connected through a pivotal coupling (543). It will be appreciated that end effector (540) and features of end effector (540), which will be described in further detail below, may be used in conjunction with end effector (140) shown in FIG. 5A. End effector (540) further comprises an outer driver (580) and a firing beam (560). Firing beam (560) comprises a blade (564) and curved portion (592). Second jaw (544) comprises a driver insert (594) operable to fit outer driver (580).

Jaws (542, 544) are operable to clamp tissue similar to jaws (142, 144). As mentioned above, jaws (542, 544) connect through pivotal coupling (543). It will be appreciated that pivotal coupling (543) is positioned below firing beam (560). It will further be appreciated that the position of pivotal coupling (543) may provide increased leverage for closing jaw (544) against jaw (542). However, it will be understood that pivotal coupling (543) may be positioned at any suitable location.

Firing beam (560) extends through shaft (530) and is operable to translate through jaws (542, 544) such that blade (564) may cut tissue that is clamped between jaws (542, 544). Firing beam (560) may be advanced similarly to firing beam (160) shown in FIG. 5A through actuation of pivoting trigger (124) by the user.

Outer driver (580) comprises a resilient longitudinal beam extending along the length of firing beam (560). Outer driver (580) is also operable to fit within driver insert (594), which forms a part of jaw (544). In particular, as firing beam (560) advances within jaws (542, 544) outer driver (580) enters driver insert (594) as seen in FIG. 13B. Outer driver (580) serves as a substitute for upper flange (62). Outer driver (580) and firing beam (560) may be coupled in any suitable manner as would be apparent to one of ordinary skill in the art in view of the teachings herein. For instance, a mortise and tenon connection (581) between outer driver (580) and firing beam (560) may be used. Alternatively, any other suitable connection operable to couple outer driver (580) and firing beam (560) may be used. It will be appreciated that outer driver (580) may comprise a deformable material such that the end entering driver insert (594) may initially deform upwardly during advancement of firing beam (560) and outer driver (580), yet the resilience of outer driver (580) drives jaw (544) toward jaw (542).

As outer driver (580) advances further along driver channel (594), jaw (544) closes further against jaw (542). It will be appreciated that the resilience of outer driver (580) may be operable to add additional clamping force to close jaw (544) against jaw (542). Furthermore, firing beam (560) also advances along jaws (542, 544), thereby transecting tissue between jaws (542, 544). Thereafter, outer driver (580) and firing beam (560) may be retracted to release tissue from jaws (542, 544). It will be understood that advancing and retracting outer driver (580) and firing beam (560) may occur using a firing beam such as firing beam (160) shown in FIG. 5A. It will further be appreciated that in some versions, any of the features described above regarding controlling inner and outer beams in stages (such as those shown in FIGS. 8A-12C) may be used to control firing beam (560) and outer driver (580) in stages. Though it will be appreciated that in some versions, firing beam (560) and outer driver (580) may simply be advanced simultaneously. When outer driver (580) and firing beam (560) are retracted, curved portion (592) cams against jaw (544) to open jaws (542, 544).

IV. Miscellaneous

It should be understood that any of the versions of electrosurgical instrument (10) described herein may include various other features in addition to or in lieu of those described above. By way of example only, any of the devices herein may also include one or more of the various features disclosed in any of the various references that are incorporated by reference herein.

It should also be understood that any of the devices described herein may be modified to include a motor or other electrically powered device to drive an otherwise manually moved component. Various examples of such modifications are described in U.S. Pub. No. 2012/0116379, entitled “Motor Driven Electrosurgical Device with Mechanical and Electrical Feedback,” published May 10, 2012, the disclosure of which is incorporated by reference herein. Various other suitable ways in which a motor or other electrically powered device may be incorporated into any of the devices herein will be apparent to those of ordinary skill in the art in view of the teachings herein.

It should also be understood that any of the devices described herein may be modified to contain most, if not all, of the required components within the medical device itself. More specifically, the devices described herein may be adapted to use an internal or attachable power source instead of requiring the device to be plugged into an external power source by a cable. Various examples of how medical devices may be adapted to include a portable power source are disclosed in U.S. Provisional Application Ser. No. 61/410,603, filed Nov. 5, 2010, entitled “Energy-Based Surgical Instruments,” the disclosure of which is incorporated by reference herein. Various other suitable ways in which a power source may be incorporated into any of the devices herein will be apparent to those of ordinary skill in the art in view of the teachings herein.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus for operating on tissue, the apparatus comprising:
(a) a body comprising an actuator;
(b) an end effector in communication with the body, wherein the end effector comprises a first jaw and a second jaw configured to clamp tissue;
(c) an outer beam configured to advance within the end effector, wherein the outer beam is configured to close the second jaw toward the first jaw, wherein the actuator is configured to advance the outer beam within the end effector; and
(d) an inner beam configured to advance within the end effector, wherein the inner beam is configured to transect tissue, wherein the actuator is configured to control the advancement of the inner beam in at least two stages, wherein the actuator is configured to advance the inner beam and the outer beam together in a first stage, wherein the actuator is configured to advance the inner beam while the outer beam remains stationary in a second stage.

2. The apparatus of claim 1, wherein the inner beam is shaped as an I-beam, wherein, the outer beam is shaped as a C-beam.

3. The apparatus of claim 2, wherein the C-beam encompasses at least part of the I-beam.

4. The apparatus of claim 1, wherein the body comprises a rack and pinion configured to advance the inner beam as the actuator is actuated.

5. The apparatus of claim 1, further comprising a spring in communication with the inner beam such that the spring applies a proximal bias to the inner beam.

6. The apparatus of claim 1, further comprising a pawl feature, wherein the inner beam comprises a pawl pocket, wherein the outer beam comprises a pawl pivot wherein the pawl feature is configured to longitudinally couple the inner beam and the outer beam, wherein the pawl feature is rotatable to disengage the inner beam from the outer beam.

7. The apparatus of claim 6, wherein the pawl feature is configured to position within the pawl pocket as the inner beam advances relative to the outer beam.

8. The apparatus of claim 7, wherein the pawl pivot is spring biased to rotate to an orientation parallel to the outer beam.

9. The apparatus of claim 6, wherein the inner beam is configured to retract relative to the outer beam to cause the pawl feature to rotate.

10. The apparatus of claim 1, wherein the outer beam is configured to advance distally ahead of the inner beam.

11. The apparatus of claim 1, further comprising a ramp feature, wherein the inner beam comprises detent feature, wherein the outer beam comprises a detent pocket, wherein the ramp is configured to lower or raise the inner beam within the end effector such that the detent feature disengages and engages the detent pocket.

12. The apparatus of claim 11, wherein the inner beam comprises an upper flange, wherein the outer beam defines a clearance for the upper flange to lower or raise within the clearance.

13. The apparatus of claim 11, wherein the inner beam comprises a lower flange operable to engage the ramp feature.

14. The apparatus of claim 11, wherein the inner beam is configured to transect at least a portion of tissue as the inner beam rides along the ramp feature.

15. The apparatus of claim 11, wherein the detent feature is configured to re-engage the detent pocket as the inner beam is retracted.

16. An apparatus comprising:
(a) a body comprising an actuator operable to be manually manipulated by a user;
(b) an end effector extending from the body, wherein the end effector is configured to grasp a portion of tissue;
(c) a first beam extending through the end effector, wherein the first beam is configured to advance within the end effector;
(d) a second beam extending through the end effector, wherein the second beam is configured to advance with the first beam when the second beam has engaged the first beam, wherein the second beam is further configured to remain longitudinally stationary when the second beam has disengaged the first beam; and
(e) an engagement feature configured to selectively engage the second beam with the first beam within the end effector.

17. The apparatus of claim 16, wherein the engagement feature comprises a pawl feature.

18. The apparatus of claim 16, wherein the engagement feature comprises a detent feature.

19. The apparatus of claim 16, wherein the first beam comprises an I-beam, wherein the second beam comprises a C-beam.

20. An apparatus for operating on tissue, the apparatus comprising:
(a) a body comprising an actuator;
(b) an end effector in communication with the body, wherein the end effector comprises a first jaw and a second jaw configured to clamp tissue; and
(c) a translating member operable to translate through the first and second jaws, wherein the translating member comprises:
(i) a vertical blade, and
(ii) an upper flange disposed above the vertical blade, wherein at least part of the upper flange is positioned to translate through a channel formed in the first jaw, wherein the upper flange is resiliently biased to drive the first jaw toward the second jaw.
